(19) United States
(12) Patent Application Publication
(10) Pub. No.: US 2026/0144748 A1
(43) Pub. Date: May 28, 2026

(54) POLYOL-BASED MULTI-FUNCTIONAL COMPOUNDS FOR MEDICAL APPLICATIONS AND HYDROGELS FORMED FROM SAME

(71) Applicant: Boston Scientific Scimed, Inc., Maple Grove, MN (US)

(72) Inventors: Rick William Dorn, Plymouth, MN (US); Joseph Thomas Delaney, Jr., Minneapolis, MN (US); Nicolas Ball-Jones, Minneapolis, MN (US); Cristian Parisi, Boston, MA (US)

(73) Assignee: Boston Scientific Scimed, Inc., Maple Grove, MN (US)

(21) Appl. No.: 19/177,927

(22) Filed: Apr. 14, 2025

Related U.S. Application Data
(60) Provisional application No. 63/633,940, filed on Apr. 15, 2024.

(51) Int. Cl. A61K 9/06; A61K 47/34

(52) U.S. Cl. CPC A61K 9/06; A61K 47/34

(57) ABSTRACT

In some aspects, the present disclosure pertains to systems for forming hydrogels that comprise: (a) a reactive multi-functional compound comprising (i) a residue of a polyol that comprises two or more hydroxyl groups and (ii) a plurality of activated ester groups, each of which is linked to the polyol residue through a residue of a cyclic anhydride and (b) a reactive polymer comprising a plurality of nucleophilic groups that are reactive with the plurality of activated ester groups of the reactive multi-functional compound. In other aspects, the present disclosure provides methods of treatment comprising administering to a subject a mixture of the reactive multi-functional compound and the multi-arm polymer to a patient. In other aspects, the present disclosure provides crosslinked hydrogels that are formed from the reactive multi-functional compound and the multi-arm polymer.

### CROSS-REFERENCE TO RELATED APPLICATIONS

This application claims the benefit of U.S. Provisional Patent application Ser. No. 63/633,940 filed on Apr. 15, 2024, the disclosure of which is incorporated herein by reference.

### FIELD

The present disclosure relates to polyol-based multi-functional compounds, to methods of forming the same, and to crosslinkable systems for forming hydrogels using such polyol-based multi-functional compounds, among other aspects. The hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups rapidly react with the trilysine crosslinker in vivo to form a hydrogel. The hydrogels breakdown in-vivo over the course of circa 6-9 months. The breakdown occurs primarily through the hydrolysis of the ester linkages on the glutarate groups.

### SUMMARY

The present disclosure provides alternative multi-functional compounds and systems for use in hydrogel products. As used herein, a “hydrogel,” also referred to as a “crosslinked hydrogel,” is a crosslinked polymer that contains water or can absorb water but does not dissolve when placed in water.

In some aspects, the present disclosure pertains to systems for forming hydrogels that comprise: (a) a reactive multi-functional compound comprising (i) a residue of a polyol that comprises two or more hydroxyl groups and (ii) a plurality of activated ester groups, each of which is linked to the polyol residue through a residue of a cyclic anhydride; and (b) a reactive polymer comprising a plurality of nucleophilic groups that are reactive with the plurality of activated ester groups of the reactive multi-functional compound.

In some aspects, the present disclosure pertains to systems for forming hydrogels that comprise: (a) a reactive multi-functional compound that is formed by a process that comprises (i) reacting hydroxyl groups of a polyol that comprises two or more of the hydroxyl groups with a cyclic anhydride compound in a ring opening reaction to form a polycarboxylic acid compound that comprises three or more carboxylic acid groups that are linked to a residue of the polyol through a hydrolysable ester group and (ii) attaching an activated ester group at a site of the carboxylic acid groups of the polycarboxylic acid compound; and (b) a reactive polymer comprising a plurality of nucleophilic groups that are reactive with the plurality of activated ester groups of the reactive multi-functional compound.

In some embodiments, which may be used in conjunction with the above aspects, the plurality of activated ester groups are cyclic imide ester groups.

In some embodiments, which may be used in conjunction with the above aspects and embodiments, the polyol is formed by reacting a polycarboxylic acid compound that comprises three or more carboxylic acid groups with a hydroxyamine compound in an amide coupling reaction.

In some embodiments, which may be used in conjunction with the above aspects and embodiments, the acid anhydride is selected from glutaric anhydride, succinic anhydride, malonic anhydride, adipic anhydride, and diglycolic anhydride.

In some embodiments, which may be used in conjunction with the above aspects and embodiments, the acid anhydride is an iodinated cyclic anhydride.

In some embodiments, which may be used in conjunction with the above aspects and embodiments, the polyol is an iodinated polyol.

In some embodiments, which may be used in conjunction with the above aspects and embodiments, the nucleophilic groups of the reactive polymer are primary amine or thiol groups.

In some embodiments, the reactive polymer is a multi-arm polymer.

In some embodiments, the reactive polymer is a polysaccharide.

In some embodiments, which may be used in conjunction with the above aspects and embodiments, the nucleophilic groups of the reactive polymer are primary amine groups, and the reactive polymer is formed from a precursor polymer that comprise a plurality of hydroxyl groups by a process in which at least a portion of the hydroxyl groups of the precursor polymer are converted to amine groups.

In some embodiments, which may be used in conjunction with the above aspects and embodiments, the nucleophilic groups of the reactive polymer are primary amine groups, and the reactive polymer is formed from a carboxylic-acid-containing precursor polymer that comprises a plurality of carboxylic acid groups by a process in which at least a portion of the carboxylic acid groups of the carboxylic-acid-containing precursor polymer are reacted in an amide coupling reaction with a single amino group of a polyamino compound that comprises two or more amino groups.

In some embodiments, which may be used in conjunction with the above aspects and embodiments, the system comprises a first composition that comprises the reactive multi-functional compound in a first container and a second composition that comprises the reactive polymer in a second container, wherein the first container and the second container are independently selected from vials and syringe barrels. In some embodiments of these embodiments, the first container is a syringe barrel and the second container is a vial.

In some embodiments, which may be used in conjunction with the above aspects and embodiments, the system further comprises a delivery device that is configured to simultaneously deliver the reactive multi-functional compound and the multi-arm polymer to a patient under conditions where the reactive multi-functional compound covalently crosslinks with the multi-arm polymer to form a hydrogel. In some of these embodiments, the delivery device is a double barrel syringe.

In other aspects, the present disclosure provides methods of treatment comprising administering to a subject a mixture of a reactive multi-functional compound and a multi-arm polymer in accordance with any of the above aspects and embodiments, wherein the mixture is administered under conditions such that the reactive multi-functional compound and the multi-arm polymer crosslink after administration to form a hydrogel.

In other aspects, the present disclosure provides crosslinked hydrogels formed by covalently crosslinking a reactive multi-functional compound and a multi-arm polymer in accordance with any of the above aspects and embodiments.

In some embodiments, the crosslinked hydrogel is in the form of crosslinked hydrogel particles. In some of these embodiments, the crosslinked hydrogel particles are contained in a syringe barrel.

In other aspects, the present disclosure provides methods of treatment comprising administering to a subject the crosslinked hydrogel of any of the above aspects and embodiments.

The above and other aspects, embodiments, features and benefits of the present disclosure will be readily apparent from the following detailed description.

### BRIEF DESCRIPTION OF THE DRAWINGS

FIG. 1 schematically illustrates a method of forming a multi-functional compound in which succinimidyl glutarate groups are linked to a glycerol residue, in accordance with an embodiment of the present disclosure.

FIG. 2 schematically illustrates a method of forming a multi-functional compound in which succinimidyl glutarate groups are linked to an erythritol residue, in accordance with an embodiment of the present disclosure.

FIG. 3 schematically illustrates a method of forming a multi-functional compound in which succinimidyl glutarate groups are linked to an iodixanol residue, in accordance with an embodiment of the present disclosure.

FIG. 4 schematically illustrates a method of forming a multi-functional compound in which succinimidyl tetraiodobenzene dicarboxylate groups are linked to a glycerol residue, in accordance with an embodiment of the present disclosure.

FIG. 5 schematically illustrates a method of forming a multi-functional compound in which succinimidyl glutarate groups are linked to an ethylene glycol-bis(2-aminoethylether)-N,N,N′,N′-tetraacetic acid residue, in accordance with an embodiment of the present disclosure.

FIG. 6 schematically illustrates a reaction for forming a crosslinked hydrogel by amide coupling between a succinimide ester group of a reactive multi-functional compound and a primary amine group of a reactive polymer, in accordance with an embodiment of the present disclosure.

FIG. 7 schematically illustrates a method of converting a hydroxy-terminated multi-arm PEG into an amino-terminated multi-arm PEG, in accordance with an embodiment of the present disclosure.

FIG. 8 schematically illustrates a method of forming a hydrogel by reaction between heparin and a multi-functional compound like that of FIG. 1, in accordance with an embodiment of the present disclosure.

FIG. 9A schematically illustrates a method of derivatizing hyaluronic acid with amino-PEG3-aminocarbonyl groups, in accordance with an embodiment of the present disclosure.

FIG. 9B schematically illustrates a method of forming a hydrogel by reaction between the modified hyaluronic acid of FIG. 9A with a multi-functional compound like that of FIG. 1, in accordance with an embodiment of the present disclosure.

FIG. 10 schematically illustrates a delivery device, in accordance with an embodiment of the present disclosure.

FIG. 11 schematically illustrates a delivery device, in accordance with another embodiment of the present disclosure.

### DETAILED DESCRIPTION

In some aspects, the present disclosure provides hydrogels that comprise a crosslinked reaction product of (a) a reactive multi-functional compound comprising a plurality of electrophilic groups and (b) a reactive polymer having a plurality of nucleophilic groups that are reactive with the electrophilic groups of the reactive multi-functional compound.

Reactive multi-functional compounds in accordance with the present disclosure may have two, three, four, five, six, seven, eight, nine, ten, eleven, twelve, thirteen, fourteen, fifteen, sixteen, seventeen, eighteen, nineteen, twenty, twenty-five, thirty or more electrophilic groups.

Electrophilic groups include activated ester groups, including cyclic imide ester groups such as succinimide ester groups,

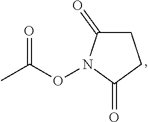

maleimide ester groups, glutarimide ester groups, diglycolimide ester groups, phthalimide ester groups, and bicyclo[2.2.1]hept-5-ene-2,3-dicarboxylic acid imide ester groups,

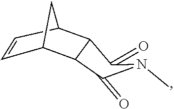

imidazole ester groups, imidazole carboxylate groups and benzotriazole ester groups, among others.

Reactive multi-functional compounds in accordance with the present disclosure may comprise a polyol core residue core and three or more electrophilic groups that are linked to the polyol residue core by a linkage that comprises a hydrolysable ester group.

In various embodiments, the reactive multi-functional compounds may be synthesized by first reacting a polyol with a cyclic anhydride in a ring opening reaction to form a polycarboxylic acid compound in which the carboxylic acid groups are linked to a residue of the polyol through a hydrolysable ester group. Then, an electrophilic group is formed at the site of each of the carboxylic acid groups of polycarboxylic acid compound. For example, in some embodiments, an N-hydroxy cyclic imide compound may be reacted with the carboxylic acid groups of the polycarboxylic acid compound in an amide coupling reaction to form a reactive multi-functional compound in which reactive cyclic imide ester groups are linked to a residue of the polyol through a linkage that comprises a hydrolysable ester group (which is part of a residue of the cyclic anhydride). In this way, a variety of multi-functional compounds can be formed.

Polyols are defined herein as compounds containing multiple hydroxyl groups. Polyols for use in the present disclosure may have two, three, four, five, six, seven, eight, nine, ten, eleven, twelve, thirteen, fourteen, fifteen, sixteen, seventeen, eighteen, nineteen, twenty, twenty-five, thirty or more hydroxyl groups. In various embodiments, polyols for use in the present disclosure may have further hydrophilic groups in addition to hydroxyl groups, including ether groups, amine groups, ester groups, amide groups.

Polyols for use in the present disclosure include non-iodinated and iodinated polyols.

Polyols may be selected, for example, from non-iodinated and iodinated sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative non-iodinated polyols also include polyhydroxylated polymers such as poly(vinyl alcohol), poly(allyl alcohol), poly(hydroxyethyl acrylate), or poly(hydroxyethyl methacrylate), among others. Such polyhydroxylated polymers may range, for example, from 3 to 100 monomer units in length.

Further illustrative non-iodinated polyols include silsesquioxanes, which are compounds that have a cage-like silicon-oxygen core that is made up of Si—O—Si linkages and tetrahedral Si vertices, exterior organic groups may be covalently attached to the cage-like silicon-oxygen core. In the present disclosure, the organic groups comprise one or more hydroxyl groups. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, Ts, T₁₀ or T₁₂ cage-like silicon-oxygen core, respectively), and where R is an organic group that comprise one or more hydroxyl groups. The Ts cage-like silicon-oxygen cores are widely studied and have the formula [RSiO_{3/2}]₈, or equivalently R₈Si₈O₁₂.

Such a structure is shown here:

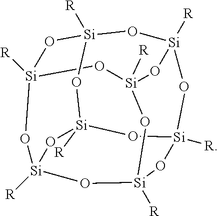

In the present disclosure, the R groups are organic groups that comprise one or more hydroxyl groups. Examples of organic groups include hydroxyalkyl groups, for example, C₁-C₄-hydroxyalkyl groups containing one, two, three or four carbon atoms and containing one, two, three or four or more hydroxyl groups, among others.

Iodinated polyols are desirable where radiopacity is desired. Iodinated polyols include iodinated aromatic polyols, examples of which are compounds that comprise two or more hydroxyl groups (e.g., two, three, four, five, six, seven, eight, nine, ten, eleven, twelve or more hydroxyl groups), and one or more iodinated aromatic groups (e.g., one, two, three, four, five, six or more iodine atoms). Examples of iodinated aromatic groups include iodine-substituted monocyclic aromatic groups and iodine-substituted multicyclic aromatic groups which may contain, for example, six, ten, fourteen, eighteen or more carbon atoms, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, amine groups, and combinations thereof, among others.

Specific examples of iodinated polyols for use in the present disclosure include commercially available

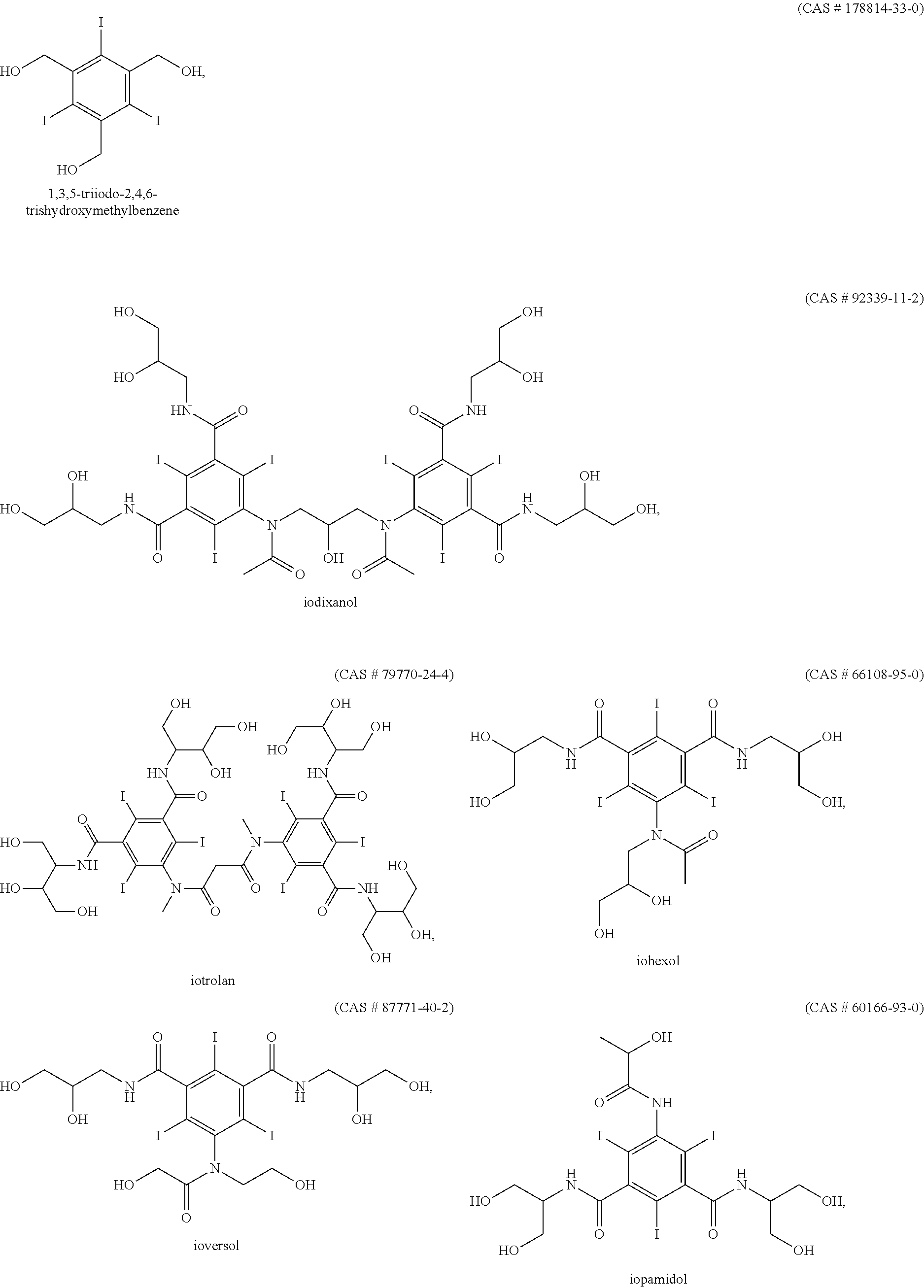 [SMILES: OCc1c(I)c(CO)c(I)c(CO)c1I ; CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I ; CN(C(=O)CC(=O)N(C)c1c(I)c(C(=O)NC(CO)C(O)CO)c(I)c(C(=O)NC(CO)C(O)CO)c1I)c1c(I)c(C(=O)NC(CO)C(O)CO)c(I)c(C(=O)NC(CO)C(O)CO)c1I ; CC(=O)N(CC(O)CO)c1c(I)c(C(=O)NCC(O)CO)c(I)c(C(=O)NCC(O)CO)c1I ; O=C(NCC(O)CO)c1c(I)c(C(=O)NCC(O)CO)c(I)c(N(CCO)C(=O)CO)c1I ; CC(O)C(=O)Nc1c(I)c(C(=O)NC(CO)CO)c(I)c(C(=O)NC(CO)CO)c1I]

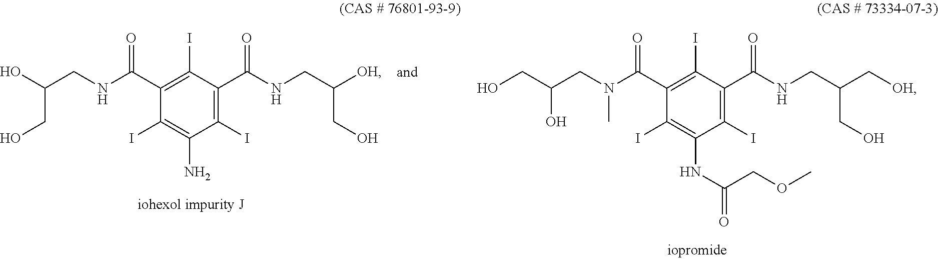 [SMILES: Nc1c(I)c(C(=O)NCC(O)CO)c(I)c(C(=O)NCC(O)CO)c1I ; COCC(=O)Nc1c(I)c(C(=O)NCC(CO)CO)c(I)c(C(=O)N(C)CC(O)CO)c1I]

among others.

Various non-iodinated and iodinated polyols, are also presented in the following table:

| | | Number of |
|---|---|---|
| Compound | CAS# | —OH Groups |
| Ethylene Glycol | 107-21-1 | 2 |
| Diethylene Glycol | 111-46-6 | 2 |
| Triethylene Glycol | 112-27-6 | 2 |
| Poly(ethylene Glycol) | | 2 |
| 5-Iodo-1,3-benzenedimethanol | 859238-49-6 | 2 |
| 2-(Hydroxymethyl)-1,3-propanediol | 4704-94-3 | 3 |
| Glycerol | 58-81-5 | 3 |
| Trimethylolethane | 77-85-0 | 3 |
| Trimethylolpropane | 77-99-6 | 3 |
| Diglycerol | 9627-82-7 | 4 |
| Erythritol | 149-32-6 | 4 |
| Pentaerythritol | 115-77-5 | 4 |
| Iopromide* | 73334-07-3 | 4 |
| 5-Acetamido-N,N′-bis(2,3- | 31127-80-7 | 4 |
| dihydroxypropyl)-2,4,6- | | |
| triiodoisophthalamide* | | |
| Metrizamide* | 31112-62-6 | 4 |
| 1,3-Benzenedicarboxamide,5- | 76350-28-2 | 4 |
| (acetylamino)-N1,N3-bis(2,3- | | |
| dihydroxypropyl)-2,4,6-triiodo-N1- | | |
| methyl- (ACI)* | | |
| Arabitol | 448-82-4 | 5 |
| Triglycerol | 20411-31-8 | 5 |
| Xylitol | 87-99-0 | 5 |
| Iopamidol* | 60166-93-0 | 5 |
| Iomeprol* | 78649-41-9 | 5 |
| 1,3-Benzenedicarboxamide,N1,N3- | 60208-45-9 | 5 |
| bis[2-hydroxy-1- | | |
| (hydroxymethyl)ethyl]-5-[(2-hydroxy- | | |
| 1-oxopropyl)amino]-2,4,6-triiodo- | | |
| (ACI)* | | |
| Ioxilan* | 107793-72-6 | 5 |
| Iopentol* | 89797-00-2 | 5 |
| Ioversol* | 87771-40-2 | 6 |
| Dipentaerythritol | 126-58-9 | 6 |
| Iohexol* | 66108-95-0 | 6 |
| Mannitol | 69-65-8 | 6 |
| Sorbitol | 50-70-4 | 6 |
| Iobitridol* | 136949-58-1 | 6 |
| Octa-(3-hydroxypropyl) POSS | 229967-81-1 | 8 |
| Tripentaerythritol | 78-24-0 | 8 |
| Iodixanol* | 92339-11-2 | 9 |
| Isomalt | 64519-82-0 | 9 |
| Maltitol | 585-88-6 | 9 |
| 1,3-Benzenedicarboxamide, 5,5′-[(1,3- | 79770-24-4 | 12 |
| dioxo-1,3- | | |
| propanediyl)bis(methylimino)]bis[N,N′- | | |
| bis[2,3-dihydroxy-1- | | |
| (hydroxymethyl)propyl]-2,4,6-triiodo- | | |
| (9CI, ACI)* | | |

*Iodinated Compounds.

As previously indicated, in various embodiments, the reactive multi-functional compounds in accordance with the present disclosure are synthesized by first reacting a polyol with a cyclic anhydride in a ring opening reaction to form a polycarboxylic acid compound in which the carboxylic acid groups are linked to a residue of the polyol through a hydrolysable ester group.

Polycarboxylic acid compounds are defined herein as compounds containing multiple carboxylic acid groups. Polycarboxylic acid compounds for use in the present disclosure may have two, three, four, five, six, seven, eight, nine, ten, eleven, twelve, thirteen, fourteen, fifteen, sixteen, seventeen, eighteen, nineteen, twenty, twenty-five, thirty or more carboxylic acid groups. Polycarboxylic acid compounds may further contain an ester group, which arises from the cyclic anhydride. More broadly, polycarboxylic acid compounds may contain one or more hydrophilic groups (e.g., those associated with a polyol residue, among others) including ether groups, amine groups, ester groups, amide groups, and other hydrophilic groups.

Cyclic anhydrides for use in with the present disclosure include non-iodinated and iodinated cyclic anhydrides.

Non-iodinated cyclic anhydrides for use in the present disclosure include, for example, succinic anhydride,

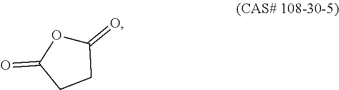

glutaric anhydride,

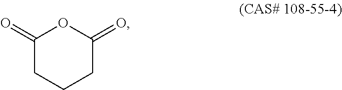

adipic anhydride,

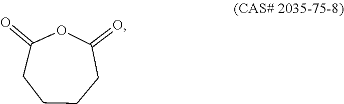

and diglycolic anhydride,

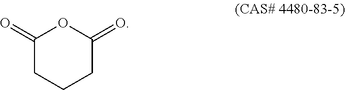

Iodinated cyclic anhydrides for use in the present disclosure include those that comprise a cyclic anhydride group and one or more iodinated aromatic groups. Examples of iodinated aromatic groups include iodine-substituted monocyclic aromatic groups and iodine-substituted multicyclic aromatic groups which may contain, for example, six, ten, fourteen, eighteen or more carbon atoms, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms.

The one or more iodinated aromatic groups may, for example, may be linked to the cyclic anhydride group though a suitable linkage (e.g., a bond or a suitable linking moiety, which may contain, for example, an alkyl group, an ether group, an amine group, an amide group, an ester group, or a combinations thereof, among others) or the one or more iodinated aromatic groups may form a multicyclic system with the cyclic anhydride group.

Examples of an iodinated cyclic anhydride in which an iodinated aromatic group is linked to cyclic anhydride group through a bond include 4-(2-iodophenyl)tetrahydropyran-2,6-dione,

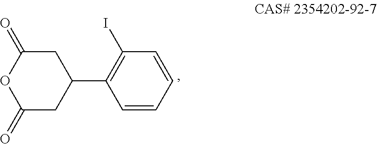 [SMILES: O=C1CC(c2ccccc2I)CC(=O)O1]

and 4-(2,3,5-triiodophenyl)tetrahydropyran-2,6-dione,

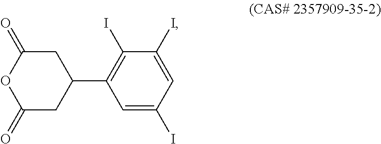 [SMILES: O=C1CC(c2cc(I)cc(I)c2I)CC(=O)O1]

among others. An example of an iodinated cyclic anhydride in which an iodinated aromatic group is linked to cyclic anhydride group through a linking group is 4-((4-iodophenyl)methyl), tetrahydropyran-2,6-dione,

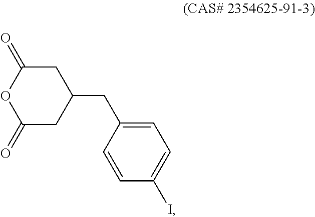 [SMILES: O=C1CC(Cc2ccc(I)cc2)CC(=O)O1]

among others. An example of an iodinated cyclic anhydride in which an iodinated aromatic group and a cyclic anhydride group form a multicyclic structure is tetraiodophthalic anhydride,

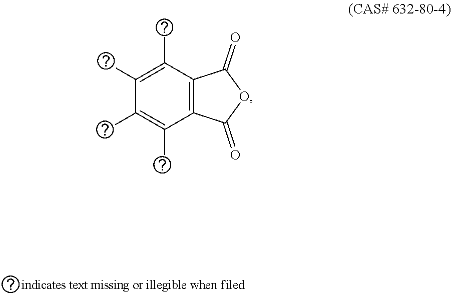

among others.

Various cyclic anhydrides, some of which are iodinated, are shown in the following table.

| Name | CAS # |
|---|---|
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |

*Iodinated

After forming a polycarboxylic acid compound, for example, as described above, a reactive moiety comprising an electrophilic group may then be linked to the polycarboxylic acid compound.

For example, in particular embodiments where a cyclic imide ester group is employed as an electrophilic group, an N-hydroxy cyclic imide compound may be reacted with the polycarboxylic acid compound in the presence of a suitable coupling agent (e.g., a carbodiimide coupling agent such as N,N′-dicyclohexylcarbodiimide (DCC), 1-ethyl-3-(3-dimethylpropyl)carbodiimide (EDC), N-hydroxybenzotriazole (HOBt), BOP reagent, and/or another coupling agent) to form an activated ester group, specifically, a reactive cyclic imide ester group that is linked to a polyol residue through a hydrolysable ester group (which corresponds to a portion of a cyclic anhydride residue). Examples of N-hydroxy cyclic imide compounds include, for example, a 1-hydroxy-2,5-pyrrolidinedione compound such as N-hydroxysuccinimide or a 1-hydroxy-3,4-substituted-2,5-pyrrolidinedione compound in which the 3-carbon and the 4-carbon form part of at least one ring in addition to the 2,5-pyrrolidinedione ring (e.g., N-hydroxy-5-norbornene-2,3-dicarboxylic acid imide, also known as N-hydroxybicyclo[2.2.1]hept-5-ene-2,3-dicarboxylic acid imide (HONB), etc.), N-hydroxymaleimide, N-hydroxyglutarimide and N-hydroxyphthalimide, among others. Examples of reactive cyclic imide ester groups formed from N-hydroxy cyclic imide compounds include, for example, a succinimide ester group, a bicyclo[2.2.1]hept-5-ene-2,3-dicarboxylic acid imide ester group, a maleimide ester group, a glutarimide ester group, and a phthalimide ester group, among others. In this way, a number of reactive diester groups can be formed.

For example, in the particular case of N-hydroxysuccinimide as an N-hydroxy cyclic imide compound, exemplary reactive diester groups include succinimidyl malonate groups, succinimidyl glutarate groups, succinimidyl succinate groups, succinimidyl adipate groups, succinimidyl diglycolate groups, and succinimidyl 1,3-acetonedicarboxylate groups (1,3-acetonedicarboxylate groups may also be referred to herein as 3-oxopentanedioate groups), among others. In the particular case of HONB as an N-hydroxy cyclic imide compound, exemplary reactive diester groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, bicyclo[2.2.1]hept-5-ene-2,3-dicarboxylic acid imidyl diglycolate groups, and bicyclo[2.2.1]hept-5-ene-2,3-dicarboxylic acid imidyl 1,3-acetonedicarboxylate groups, among others. In the particular case of N-hydroxymaleimide as an N-hydroxy cyclic imide compound, exemplary reactive diester groups include maleimidyl malonate groups, maleimidyl glutarate groups, maleimidyl succinate groups, maleimidyl adipate groups, maleimidyl diglycolate groups, and maleimidyl 1,3-acetonedicarboxylate groups, among others. In the particular case of N-hydroxyglutarimide as an N-hydroxy cyclic imide compound, exemplary reactive diester groups include glutarimidyl malonate groups, glutarimidyl glutarate groups, glutarimidyl succinate groups, glutarimidyl adipate groups, glutarimidyl diglycolate groups, and glutarimidyl 1,3-acetonedicarboxylate groups, among others. In the particular case of N-hydroxyphthalimide as an N-hydroxy cyclic imide compound, exemplary reactive diester groups include phthalimidyl malonate groups, phthalimidyl glutarate groups, phthalimidyl succinate groups, phthalimidyl adipate groups, phthalimidyl diglycolate groups, and phthalimidyl 1,3-acetonedicarboxylate groups, among others.

Thus, reactive multi-functional compounds in accordance with the present disclosure may be synthesized by first reacting a polyol with a cyclic anhydride in a ring opening reaction to form a polycarboxylic acid compound in which the carboxylic acid groups are linked to a residue of the polyol through a hydrolysable ester group. Cyclic anhydrides for use in with the present disclosure include non-iodinated and iodinated cyclic anhydrides, which may be selected from those described above. A reactive moiety comprising an electrophilic group may then be linked to the polycarboxylic acid compound. For example, in particular embodiments where a cyclic imide ester group is employed as an electrophilic group, an N-hydroxy cyclic imide compound, which may be selected from those described above, is reacted with the polycarboxylic acid compound

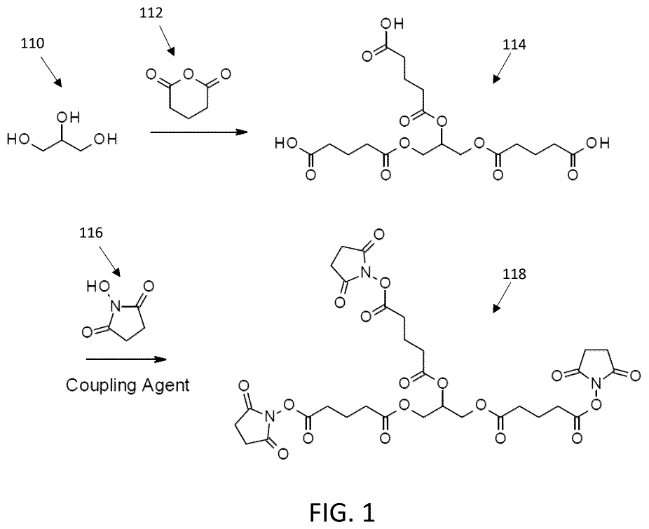

In one example shown in FIG. 1, a non-iodinated polyol, specifically, glycerol (110), is used to ring open a non-iodinated cyclic anhydride, specifically, glutaric anhydride (112) to form a polycarboxylic acid compound (114) having three carboxylic groups, in which each of the carboxylic acid groups is linked to residue of the polyol through a hydrolysable ester group. The polycarboxylic acid compound (114) is then reacted with an N-hydroxy cyclic imide compound, specifically, N-hydroxysuccinimide (116), in the presence of a suitable coupling agent, such as a carbodiimide coupling agent, to yield a multi-functional compound (118) in which cyclic imide diester groups, specifically, succinimidyl glutarate groups, are attached to a polyol residue, in particular, a glycerol residue, at the three positions corresponding the hydroxyl groups of the polyol.

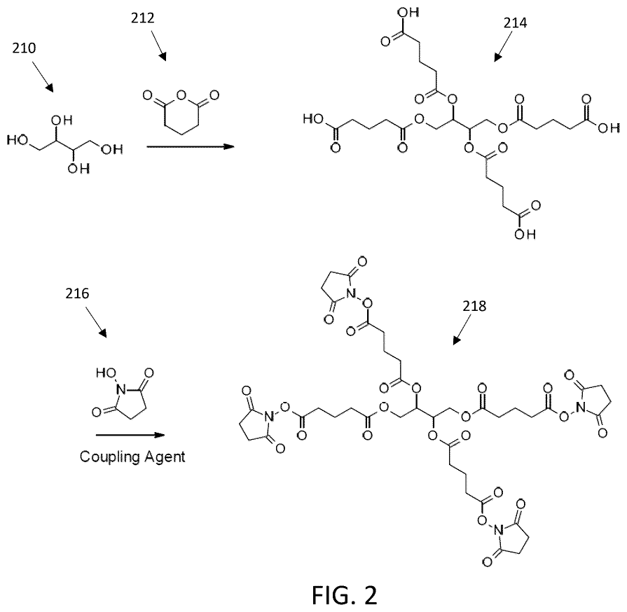

In another particular example shown in FIG. 2, a non-iodinated polyol, specifically, erythritol (210), is used to ring open a non-iodinated cyclic anhydride, specifically, glutaric anhydride (212) to form a polycarboxylic acid compound (214) having four carboxylic groups, in which each of the carboxylic acid groups is linked to a residue of the polyol through a hydrolysable ester group. The polycarboxylic acid compound (214) is then reacted with an N-hydroxy cyclic imide compound, specifically, N-hydroxysuccinimide (216), in the presence of a suitable coupling agent, to yield a multi-functional compound (218) in which cyclic imide diester groups, specifically, succinimidyl glutarate groups, are attached to a polyol residue, specifically an erythritol residue, at the four positions corresponding to the hydroxyl groups of the polyol.

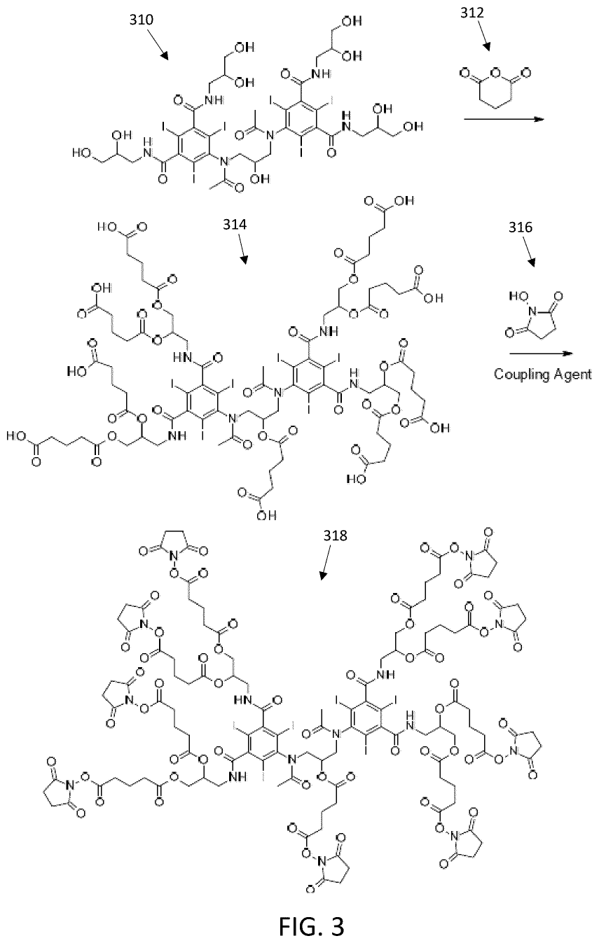

In a further particular example shown in FIG. 3, an iodinated polyol, specifically, iodixanol (310), is used to ring open a non-iodinated cyclic anhydride, specifically, glutaric anhydride (312) to form a polycarboxylic acid compound (314) having nine carboxylic groups, in which each of the carboxylic acid groups is linked to a residue of the iodixanol through a hydrolysable ester group. The polycarboxylic acid compound (314) is then reacted with an N-hydroxy cyclic imide compound, specifically, N-hydroxysuccinimide (316), in the presence of a suitable coupling agent, to yield a multi-functional compound (318) in which cyclic imide diester groups, specifically, succinimidyl glutarate groups, are attached to a polyol residue, specifically an iodixanol residue, at the nine positions corresponding to the hydroxyl groups of the polyol.

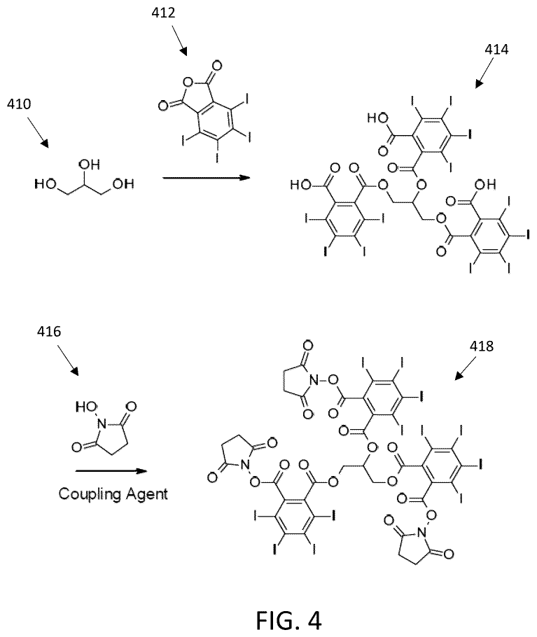

In yet another particular example shown in FIG. 4, a non-iodinated polyol, specifically, glycerol (410), is used to ring open an iodinated cyclic anhydride, specifically, tetraiodophthalic anhydride (412) to form a polycarboxylic acid compound (414) having three carboxylic groups, in which each of the carboxylic acid groups is linked to residue of the polyol through an iodinated aromatic group and a hydrolysable ester group. The polycarboxylic acid compound (114) is then reacted with an N-hydroxy cyclic imide compound, specifically, N-hydroxysuccinimide (416), in the presence of a suitable coupling agent, to yield a multi-functional compound (418) in which cyclic imide ester groups, specifically, succinimidyl tetraiodobenzene dicarboxylate groups, are attached to a polyol residue, specifically a glycerol residue, at the three positions corresponding to the hydroxyl groups of the polyol.

In some embodiments, a polycarboxylic acid compound may be reacted with a hydroxyamine compound in an amide coupling reaction in the presence of a suitable coupling agent, such as a carbodiimide coupling agent, to form a polyol that contains multiple hydroxyl groups and multiple amide groups, that correspond in number to the number of carboxylic acid groups in the polycarboxylic acid compound.

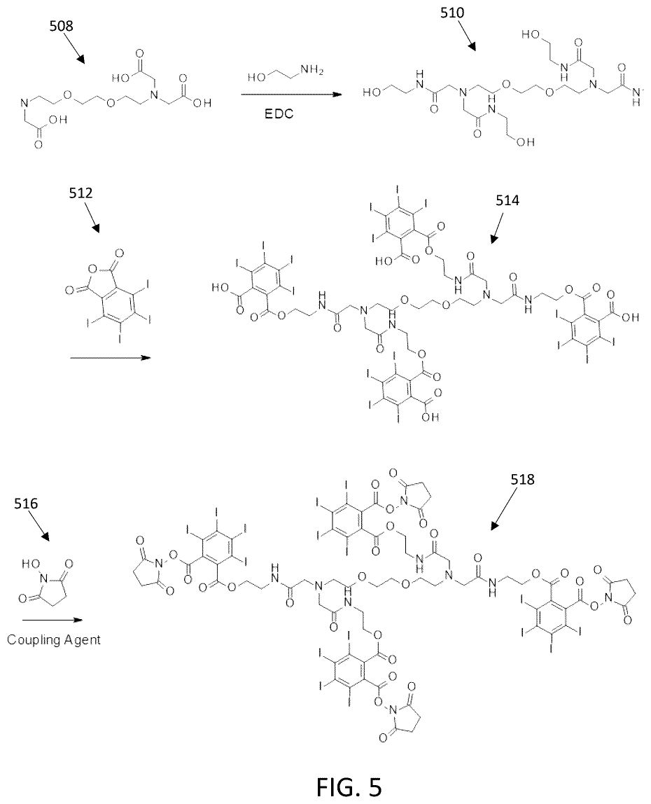

In a particular example shown in FIG. 5, a polycarboxylic acid compound, specifically, a polycarboxylic acid polyether compound, more specifically, ethylene glycol-bis(2-aminoethylether)-N,N,N′,N′-tetraacetic acid (EGTA) (CAS #67-42-5) (508) is reacted with a hydroxyamine compound, for example, a C₁-C₄-hydroxyamine compound such as ethanolamine, in an amide coupling reaction in the presence of a carbodiimide coupling agent, specifically, EDC, to form a polyol (510) in which C₁-C₄-hydroxyalkyl groups are linked to a polycarboxylic acid compound residue, specifically, an EGTA residue, through amide groups. The polyol (510) is then used to form an ester-containing, radiopaque, activated ester crosslinker. In particular, the polyol (510) is reacted in a ring opening reaction with an iodinated cyclic anhydride, specifically, tetraiodophthalic anhydride (512) to form a polycarboxylic acid compound (514) having four carboxylic groups, in which each of the carboxylic acid groups is linked to residue of the polyol (510) through a linkage that comprises an iodinated group and a hydrolysable ester group. The polycarboxylic acid compound (514) is then reacted with an N-hydroxy cyclic imide compound, specifically, N-hydroxysuccinimide (516), in the presence of a suitable coupling agent, to yield a multi-functional compound (518) in which cyclic imide ester groups, specifically, succinimidyl tetraiodobenzene dicarboxylate groups, are attached to residue of polyol (510) at the four positions corresponding to the hydroxyl groups of the polyol (510).

In some aspects, the present disclosure provides hydrogels that comprise a crosslinked reaction product of (a) a reactive multi-functional compound comprising a plurality of electrophilic groups as described above and (b) a reactive polymer having a plurality of nucleophilic groups that are reactive with the electrophilic groups of the reactive multi-functional compound.

Nucleophilic groups may be selected, for example, amine groups, particularly primary amine and thiol groups.

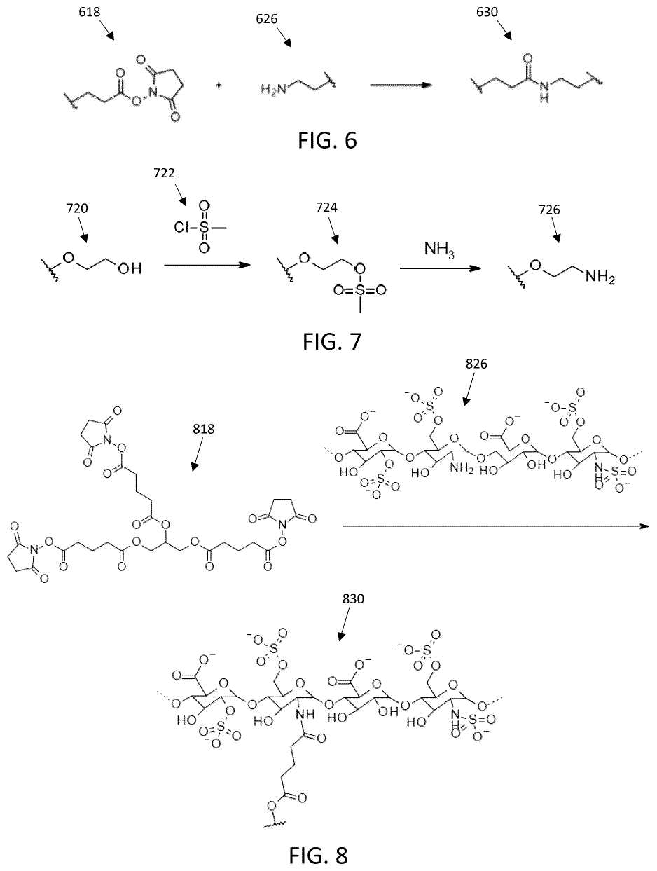

FIG. 6 schematically illustrates an amide coupling reaction between an activated ester group, specifically, a succinimide ester group, of a reactive multi-functional compound (618) (only a single succinimide ester group is illustrated; the remainder of the reactive multi-functional compound (618) is not illustrated) and a primary amine group of a reactive polymer (626) having a plurality of primary amine groups (only a single primary amine group is illustrated; the remainder of the reactive polymer (626) is not illustrated)) whereby a crosslinked hydrogel (630) is formed (only a single amide linkage formed from the reaction between the succinimide ester group and the primary amine group is shown; the remainder of the crosslinked hydrogel (630) is not illustrated).

The crosslinked hydrogels of the present disclosure may be formed in vivo (e.g., using a delivery device like that described below), or such crosslinked hydrogels may be formed ex vivo and subsequently administered to a subject. The crosslinked hydrogels of the present disclosure may be used in a variety of biomedical applications, including implants, medical devices, and pharmaceutical compositions.

In various embodiments, the crosslinked hydrogels are visible under fluoroscopy. The crosslinked hydrogels may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on bench-top micro-CT systems such as XtremeCT from Scanco Medical (Wangen-Brittisellen, Switzerland) or similar.

In some embodiments, the hydrogels break down in vivo over a period ranging from 1 day or less to 5 years or longer, depending on the desired application, for example, ranging anywhere from 1 day to 3 days to 1 week to 2 weeks to 1 month to 6 months to 1 year to 2 years to 5 years of longer (in other words, over a period ranging between any two of the preceding values).

In some aspects, the present disclosure provides systems that comprise (a) a reactive multi-functional compound comprising a plurality of electrophilic groups as described above and (b) a reactive polymer having a plurality of nucleophilic groups that are reactive with the electrophilic groups of the reactive multi-functional compound.

Reactive polymers for use in the present disclosure include amine-functionalized hydrophilic polymers that contain one or more hydrophilic polymer segments and two or more amine groups, for example, ranging anywhere from 2 to 3 to 4 to 5 to 6 to 7 to 8 to 9 to 10 to 11 to 12 to 13 to 15 to 20 to 25 to 50 to 75 to 100 amine groups (in other words, having a number of amine groups ranging between any two of the preceding values).

In some embodiments, the reactive polymers are reactive multi-arm polymers that comprise a plurality of polymer arms linked to a core region, wherein each of the arms comprises a hydrophilic polymer segment and wherein each of the arms comprises a terminal nucleophilic group.

Reactive polymers in accordance with the present disclosure include polymers having from 2 to 100 arms, for example ranging anywhere from 2 to 3 to 4 to 5 to 6 to 7 to 8 to 9 to 10 to 11 to 12 to 13 to 15 to 20 to 25 to 50 to 75 to 100 arms (in other words, having a number of arms ranging between any two of the preceding values).

The at least one terminal nucleophilic group may be linked to the hydrophilic polymer segment through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 2 and 1000 monomer units or more, for example, ranging anywhere from 2 to 3 to 4 to 6 to 8 to 10 to 15 to 20 to 25 to 50 to 100 to 250 to 500 to 1000 monomer units (in other words range between any two of the preceding values).

In certain embodiments, the core region comprises a residue of a polyol comprising three or more hydroxyl groups, which is used to form the polymer arms. In certain beneficial embodiments, the core region comprises a residue of a polyol that contains from 2 to 100 hydroxyl groups, for example, ranging 2 to 3 to 4 to 5 to 6 to 8 to 10 to 15 to 20 to 25 to 50 to 75 to 100 hydroxyl groups. Illustrative polyol residues may be selected, for example, from residues of the non-iodinated and iodinated polyols described above for use in forming the reactive multi-functional compounds of the present disclosure.

Reactive polymers in accordance with the present disclosure can be formed from polymers that contain primary hydroxyl groups, carboxylic acid groups, and methyl ester groups, among others, including hydroxy-terminated polymers such as hydroxy-terminated multi-arm polymers, carboxylic-acid-terminated polymers such as carboxylic-acid-terminated multi-arm polymers, and methyl-ester-terminated polymers such as methyl-ester-terminated multi-arm polymers.

In a particular example shown in FIG. 7, a hydroxy-terminated polymer, for example, a multi-arm polymer having arms that comprise one or more hydroxyl end groups, such as a hydroxy-terminated multi-arm polyethylene glycol (PEG) (720) (only a single hydroxyl group is illustrated; the remainder of the hydroxy-terminated polymer is not illustrated) is first treated with methanesulfonyl chloride (CAS #124-63-0) to form an intermediate methanesulfonate-terminated multi-arm polymer (724) in which hydroxyl groups of the hydroxy-terminated polymer are converted into methanesulfonate groups (only a single methanesulfonate group is illustrated; the remainder of the methanesulfonate-terminated polymer is not illustrated). The methanesulfonate groups are then reacted with ammonia to form an amino-terminated multi-arm polymer (726) in which methanesulfonate groups of the methanesulfonate-terminated multi-arm PEG (724) are converted into amino groups (only a single amino group is illustrated; the remainder of the amino-terminated polymer is not illustrated).

In other embodiments, the reactive polymer is a polymer that contains primary amine containing monomers such as allyl amine (e.g., poly(allyl amine) polymers and copolymers), vinyl amine (e.g., poly(vinyl amine) polymers and copolymers), and glucosamine polymers (e.g. heparin, chitosan, etc.).

Turning to FIG. 8, a reactive polymer having a plurality of nucleophilic groups, specifically, heparin (826), which contains primary amine groups, is reacted with a reactive multi-functional compound comprising a plurality of electrophilic groups, specifically, the multi-functional compound (818) of FIG. 1, in which succinimidyl glutarate groups are linked to a glycerol residue, with the multi-functional compound (818) acting as a crosslinking agent for the heparin, thereby forming a hydrogel (830). In FIG. 8, only a portion of a single chain of the crosslinked heparin and only a single succinimidyl glutarate reside of the single multi-functional compound are illustrated.

In other embodiments, the reactive polymer is formed from a polymer that does not contains primary amine groups but rather contains carboxylic acid groups, which are used to form primary amine groups. Examples of such polymers include carboxylic-acid-containing polysaccharides that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose.

In some instances, the transformation from carboxylic acid groups to primary amine groups is carried out with a polyamino compound having n amino groups, where n is two, three, four, or more, in which all but one of the amino groups have been protected with a suitable protecting agent. For example, all but one of the n amino groups of the polyamino compound can be reacted with a suitable protective agent such as di-tert-butyl dicarbonate (Boc₂O), benzyl chloroformate or fluorenylmethyloxycarbonyl chloride to form a polyamino compound in which 1 amino group remain unprotected, while the remaining n−1 amino groups are boc-protected. The unprotected amino group this boc-protected polyamino compound can then be reacted in an amide coupling reaction with carboxylic acid groups of carboxylic-acid-containing polymer in the presence of a suitable coupling agents such as a carbodiimide coupling agent.

Examples of polyamino compounds which may be protected in this fashion include ethylene diamine, 1,3-propanediamine, amino-PEG3-amine (CAS #929-75-9), tris(3-aminopropyl)amine, 3-(2-aminoethyl)pentane-1,5-diamine, N,N′,N′-tetrakis(2-aminoethyl)-1,2-ethanediamine, 1,3,5-tris-(2-aminoethyl)-[1,3,5]triazinane-2,4,6-trione, N,N,N′-Tris(2-aminoethyl)ethylenediamine, and adamantane-13,5,7-tetraamine, among many others.

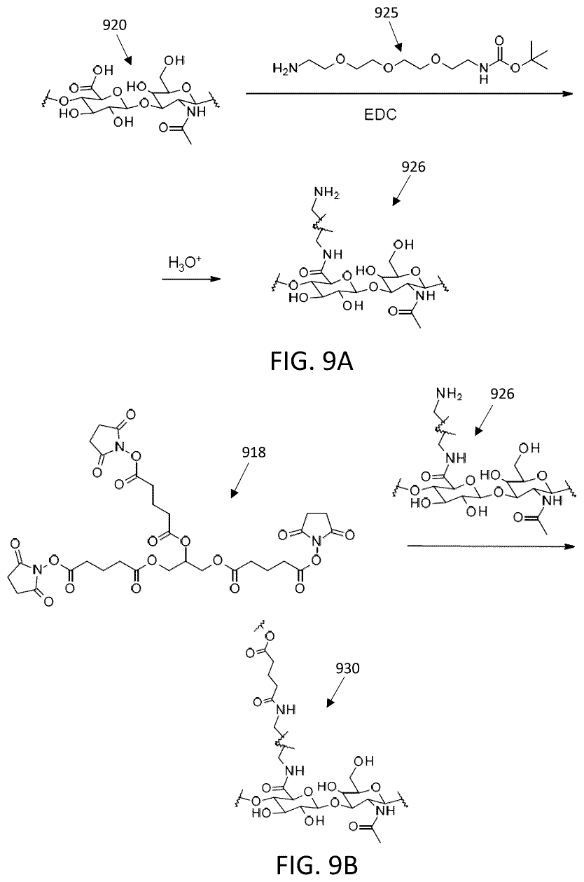

Turning now to FIG. 9A, a carboxylic-acid-containing polysaccharide, specifically hyaluronic acid (920) is coupled with amino-PEG3-amine in which one of the amino groups is boc-protected and the other amino group is unprotected, specifically, 1,1-dimethylethyl 13-amino-5,8,11-trioxa-2-azatridecanoate (925) (CAS #101187-40-0), in the presence of a carbodiimide coupling agent, specifically, EDC, followed by acid deprotection to form a primary-amine-derivatized carboxylic-acid-containing polysaccharide, specifically, amino-PEG3-aminocarbonyl substituted hyaluronic acid (926) in which at least a portion of the carboxylic acid groups have been converted to amino-PEG3-aminocarbonyl groups. In FIG. 9A, only a portion of the hyaluronic acid and only a portion of the amino-PEG3-aminocarbonyl group is illustrated.

Turning to FIG. 9B, a reactive polymer having a plurality of nucleophilic groups, specifically, the amino-PEG3-aminocarbonyl substituted hyaluronic acid (926) of FIG. 9A is reacted with a reactive multi-functional compound comprising a plurality of electrophilic groups, specifically, the multi-functional compound (818) of FIG. 1, in which succinimidyl glutarate groups are linked to a glycerol residue, with the multi-functional compound (818) acting as a crosslinking agent for the amino-PEG3-aminocarbonyl substituted hyaluronic acid (926), thereby forming a hydrogel (830). In FIG. 9B, only a portion of a single chain of the substituted hyaluronic acid and only a single succinimidyl glutarate reside of a single multi-functional compound are illustrated.

As seen from the above, by using ester-containing activated-ester crosslinkers, bioerodible hydrogels can be formed with hydrophilic polymers that are not fully bioerodible on their own, such as PEG, heparin and hyaluronic acid, among others. In some embodiments, lower molecular weight (e.g., ranging from 1,000 to 5,000 to 10,000 to 50,000 to 100,000 to 500,000 or more Da, depending on the polymeric system) versions of the hydrophilic polymers are employed so that they can be fully water soluble and non-gelling on their own in bodily fluids after being released upon hydrolysis of the ester groups within the crosslinks.

In some aspects of the present disclosure, systems are provided that are configured to deliver (a) reactive multi-functional compound as described herein and (b) a reactive polymer as described herein. The reactive multi-functional compound and reactive polymer are comingled under conditions such that electrophilic groups of the reactive multi-functional compound react and form covalent bonds with nucleophilic groups of the reactive polymer. Such systems can be used to form crosslinked hydrogels, either in vivo or ex vivo.

In some aspects of the present disclosure, systems are provided that comprise (a) a first composition that comprises a reactive multi-functional compound as described herein and (b) a second composition that comprises a reactive polymer as described herein. For example, the first and second compositions can be first and second fluid compositions that, when the first and second fluid compositions are mixed, lead to covalent bond formation between the reactive multi-functional compound and the reactive polymer, resulting in a crosslinked reaction product of the reactive multi-functional compound and the reactive polymer.

In some embodiments, systems are provided that comprise (a) a first composition containing the reactive multi-functional compound and the reactive polymer and (b) a second composition comprising an accelerant that accelerates a crosslinking reaction between the reactive multi-functional compound and the reactive polymer. For example, the first composition may be a fluid composition in which the reactive multi-functional compound and the reactive polymer are intermixed under conditions where crosslinking is suppressed between electrophilic groups of the reactive multi-functional compound and nucleophilic groups of the reactive polymer, and the second composition may be a fluid composition that, when mixed with the first fluid composition, causes covalent bonds to form between the reactive multi-functional compound and the reactive polymer, resulting in a crosslinked reaction product of the reactive multi-functional compound and the reactive polymer. In certain embodiments, the accelerant in the second fluid composition changes the pH of the first fluid composition, resulting in crosslinking between the reactive multi-functional compound and the reactive polymer.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a reactive multi-functional compound as described above and a second reservoir that contains a second fluid composition that comprises a reactive polymer as described above. When the first and second fluid compositions are mixed, crosslinking occurs between the reactive multi-functional compound and the reactive polymer.

In some embodiments, the system may include a delivery device that comprises a first reservoir that contains a first fluid composition that comprises the reactive multi-functional compound and the reactive polymer, and a second reservoir that contains a second fluid composition that is an accelerant composition. The second fluid composition, when mixed with the first fluid composition, results in crosslinking between the reactive multi-functional compound and the reactive polymer.

In either case, during operation, the first fluid composition and second fluid composition are dispensed from the first and second reservoirs and combined, whereupon the reactive polymer and the reactive multi-functional compound and crosslink with one another to form a crosslinked reaction product, specifically, a crosslinked hydrogel.

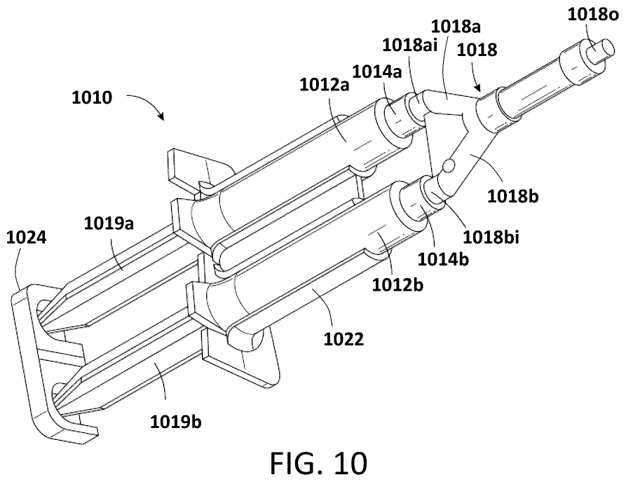

In particular embodiments, and with reference to FIG. 10, the system may include a delivery device 1010 that comprises a double-barrel syringe, which includes a first barrel 1012a having a first barrel outlet 1014a, which first barrel contains the first fluid composition, a first plunger 1016a that is movable in the first barrel 1012a, a second barrel 1012b having a second barrel outlet 1014b, which second barrel 1012b contains the second fluid composition, and a second plunger 1016b that is movable in the second barrel 1012b. In some embodiments, the device 1010 may further comprise a mixing section 1018 having a first mixing section inlet 1018ai in fluid communication with the first barrel outlet 1014a, a second mixing section inlet 1018bi in fluid communication with the second barrel outlet, and a mixing section outlet 1018o. Also shown are a syringe holder 1022 configured to hold the first and second syringe barrels 1012a, 1012b, in a fixed relationship and a plunger cap 1024 configured to hold the first and second plungers 1016a, 1016b in a fixed relationship.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

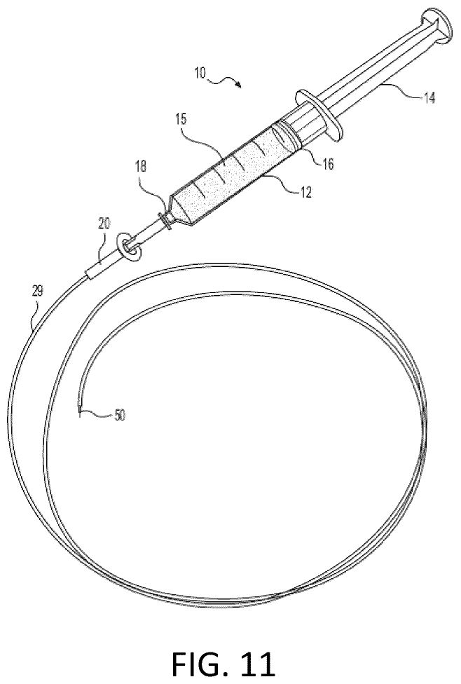

FIG. 11 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

The crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the crosslinked hydrogel compositions of the present disclosure can be imaged using a suitable imaging technique.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a hydrogel comprising:
a reactive multi-functional compound comprising (i) a residue of a polyol that comprises two or more hydroxyl groups and (ii) a plurality of activated ester groups, each of which is linked to the polyol residue through a residue of a cyclic anhydride; and
a reactive polymer comprising a plurality of nucleophilic groups that are reactive with the plurality of activated ester groups of the reactive multi-functional compound.

2. The system of claim 1, wherein the plurality of activated ester groups are cyclic imide ester groups.

3. The system of claim 1, wherein the polyol is formed by reacting an additional polycarboxylic acid compound that comprises three or more carboxylic acid groups with a hydroxyamine compound in an amide coupling reaction.

4. The system of claim 1, wherein the acid anhydride is selected from glutaric anhydride, succinic anhydride, malonic anhydride, adipic anhydride, and diglycolic anhydride.

5. The system of claim 1, wherein the acid anhydride is an iodinated cyclic anhydride and/or wherein the polyol is an iodinated polyol.

6. The system of claim 1, wherein the reactive polymer is a multi-arm polymer or the reactive polymer is a polysaccharide.

7. The system of claim 1, wherein the nucleophilic groups are primary amine or thiol groups.

8. The system of claim 7, wherein the reactive polymer is formed from a precursor polymer that comprises a plurality of hydroxyl groups by a process in which at least a portion of the hydroxyl groups of the precursor polymer are converted to amine groups.

9. The system of claim 7, wherein the reactive polymer is formed from a carboxylic-acid-containing precursor polymer that comprises a plurality of carboxylic acid groups by a process in which at least a portion of the carboxylic acid groups of the carboxylic-acid-containing precursor polymer are reacted in an amide coupling reaction with a single amino group of a polyamino compound that comprises two or more amino groups.

10. The system of claim 1, comprising a first composition that comprises the reactive multi-functional compound in a first container and a second composition that comprises the reactive polymer in a second container, wherein the first container and the second container are independently selected from vials and syringe barrels.

11. The system of claim 10, wherein the first container is a syringe barrel and the second container is a vial.

12. The system of claim 1, further comprising a delivery device that is configured to simultaneously deliver the reactive multi-functional compound and the multi-arm polymer to a patient under conditions where the reactive multi-functional compound covalently crosslinks with the multi-arm polymer to form a hydrogel.

13. The system of claim 12, wherein the delivery device is a double barrel syringe.

14. A system for forming a hydrogel comprising:
a reactive multi-functional compound that is formed by a process that comprises (a) reacting hydroxyl groups of a polyol that comprises two or more of the hydroxyl groups with a cyclic anhydride compound in a ring opening reaction to form a polycarboxylic acid compound that comprises three or more carboxylic acid groups that are linked to a residue of the polyol through a hydrolysable ester group and (ii) attaching an activated ester group at a site of the carboxylic acid groups of the polycarboxylic acid compound; and
a reactive polymer comprising a plurality of nucleophilic groups that are reactive with the plurality of activated ester groups of the reactive multi-functional compound.

15. The system of claim 14, wherein the activated ester group is a cyclic imide ester group.

16. The system of claim 15, wherein the process comprises reacting an N-hydroxy cyclic imide compound with the carboxylic acid groups of the polycarboxylic acid compound in an amide coupling reaction to form the reactive multi-functional compound.

17. A method of treatment using the system of claim 1, the method comprising administering to a subject a mixture of the reactive multi-functional compound and the multi-arm polymer, wherein the mixture is administered under conditions such that the reactive multi-functional compound and the multi-arm polymer crosslink after administration to form a hydrogel.

18. A crosslinked hydrogel formed by covalently crosslinking (a) a reactive multi-functional compound comprising (i) a residue of a polyol that comprises two or more hydroxyl groups and (ii) a plurality of activated ester groups, each of which is linked to the polyol residue through a residue of a cyclic anhydride and (b) a reactive polymer comprising a plurality of nucleophilic groups that are reactive with the plurality of activated ester groups of the reactive multi-functional compound.

19. The crosslinked hydrogel of claim 18, wherein the crosslinked hydrogel is in the form of crosslinked hydrogel particles.

20. A method of treatment comprising administering to a subject the crosslinked hydrogel of claim 18.
